(19) United States
(12) Patent Application Publication
(10) Pub. No.: US 2014/0080149 A1
(43) Pub. Date: Mar. 20, 2014

(54) Method and Arrangement for Quantifying Subgroups from a Mixed Population of Cells

(71) Applicant: Wolfgang Goehde, Nottuln (DE)

(72) Inventor: Wolfgang Goehde, Nottuln (DE)

(21) Appl. No.: 14/085,416

(22) Filed: Nov. 20, 2013

Related U.S. Application Data
(63) Continuation of application No. PCT/DE2012/100144, filed on May 16, 2012.

(30) Foreign Application Priority Data
May 20, 2011 (DE) 10 2011 050 550.4

(51) Int. Cl. G01N 33/569

(52) U.S. Cl. CPC G01N 33/56972
USPC 435/7.24; 435/287.2

(57) ABSTRACT

A method for determining the mass and concentration of certain particles or cells from a mixed population of cells, such as a patient's blood sample. The cells to be determined are each marked with a monoclonal antibody, to which colloidal iron is coupled. The blood sample is filled into a test tube with a capillary section and a magnet is used to gather the marked cells and move them to the capillary section. A test tube rack is constructed to hold the test tube with the capillary section and a measurement scale is provided for determining the mass and concentration of the marked cells.

### BACKGROUND INFORMATION

1. Field of the Invention

The invention relates to the field of laboratory blood tests. More particularly, the invention relates to a method of determining a count and percentage of CD4-positive cells in a blood sample taken from a patient.

2. Discussion of the Prior Art

In cell research, medical diagnostics, and with microbial analysis, it is often necessary, to quantify the proportion of certain subgroups of cells in mixed cell populations.

Immune diagnostics is a particularly widespread field of application. In this case, the concentration of certain subgroups of white blood cells that have been marked with immune markers is to be determined. The precise determination of the concentration of such cells in the blood of sick persons or treated patients enables the development of a precise and individualized therapy plan. This concept has acquired particular importance for the treatment of HIV/AIDS patients.

In this situation, the concentration of the so-called CD4-positive T helper cells is ascertained during therapy and, indeed, lifelong. This value is then the basis for the decision for the life-sustaining therapy. The CD4-positive lymphocytes are on the one hand the cells that are preferably destroyed by the HI virus and, on the other hand, responsible for the immune defenses in the body.

The constant recording—typically four times per year—of the concentration of the CD4-positive lymphocytes is absolutely necessary for the life-sustaining therapy.

The great number of persons affected worldwide (currently almost 40 million HIV positive persons) means that very special demands are placed on the methods for determining the CD4 cell count. The majority of the infected persons lives in countries or regions with particularly deficiently equipped clinic and laboratory infrastructure. Frequently the infected persons cannot reach the service facilities that are able to determine the CD4 cell count because of lack of transportation or because the distance is too great. The necessary lab work depends on the use of highly complex measurement apparatus, that can only be operated in labs with high technical standards. These apparatuses are operated by highly specialized experts. As a result of the complexity of these requirements, the majority of HIV/AIDS patients that need this test in order to work up a successful treatment plan are not recorded. Currently only about 10% of the infected persons worldwide are being treated.

3. The State of the Art

At this time, the most widespread and scientifically the best funded method for determining the CD4-positive lymphocytes in the blood of an HIV infected person is flow-through cytophotometry. This equipment is extremely expensive and it must be operated in a laboratory that has a particularly high standard. The so-called “point of care” diagnostic, by which one brings the service directly to the affected person, does not work.

The measurement with this flow-through cytophotometry requires that the blood of the patient is incubated with certain monoclonal antibodies, in this case: CD4 monoclonal antibodies. The CD4 antibody, which has previously been marked with a fluorescent dye, binds with CD4 antigen of the cell that are always at the cell membrane. If this pre-treated blood sample is transported through a flow-through cytophotometer, all cells that carry the CD4 antigen on their cell membranes send out a fluorescent signal. Thus it is possible to achieve an exact count of the CD4-positive cells and their concentration.

In another method that has been put into practice, the blood samples is incubated in the same manner with the corresponding CD4 antibodies and then the number of the fluorescing cells is statically determined, i.e., for example, on a microscope object carrier using an image analysis process. This technique, too, requires significant effort in the way of equipment, it is expensive, and it has to be done by specially trained experts.

New, simple, less expensive methods are constantly being sought, so that the diagnostics necessary to develop effective treatment plans can reach more people. Furthermore, methods are sought so that truly “point of care” diagnostics can be brought to where the patients live.

What is needed, therefore, is a particularly simple, rugged, service-free, and inexpensive method and the corresponding evaluation unit that can replace the complex and expensive apparatus that is used to date in determining cell counts of subgroups in a mixed cell population.

### BRIEF SUMMARY OF THE INVENTION

The method according to the invention includes steps for preparing a blood sample, separating out at least one subgroup of cells of interest, and obtaining a reliable information as to cell count and percentage of cells of interest that make up the blood. The invention also includes technically simple tools to facilitate the separation and counting of cells of interest that allow the method to be used as a “point of care” delivery method of providing care to patients at locations close to where the patients are. In the description that follows, the cells of particular interest are CD4-positive cells and other cells of interest include monocytes and CD4-positive T-lymphocytes. Generally, the term “cells of interest” is used to refer to any of these groups that are to be separated out from the rest of the cells in the blood sample.

The steps of the method according to the invention for preparing the patient's blood for analysis are as follows:

A CD4 monoclonal antibody is marked with colloidal iron, added to the blood sample, and the sample incubated for a short period of time. The sample is then filled into a narrow tube and those cells to which the CD4 antibodies with the iron particles have attached, i.e., the cells of interest, are then moved to the end of the tube by passing a magnet along the tube. All other blood cells are not moved by the magnet. Now that the cells of interest have been separated out, their total volume and also the portion of the volume of blood that these cells make out can be read by on a graduated scale.

The tools to implement the inventive method are very simple and include a test tube, in which the length of the tube has a section with a wide diameter and a continuing section with a narrow, capillary diameter, and a magnet. To collect the marked cells, the magnet is moved along the tube in the axial direction, from the wide end of the tube to the narrow end, thereby gathering and holding the cells of interest at the location of the magnet, in this case, in the narrow end.

The method also include steps to improve the readability of test results. In this case, the blood is marked with two different antibodies: one carrying the iron particle and a second antibody that also marks the CD4 antigen and which is linked with a fluorescent dye. After incubating the blood, the iron-particle-carrying CD4-positive cells are moved to the end of the tube with the magnet and the cells then illuminated with a light that excites the fluorescence, thereby making the cells of interest clearly visible.

In addition to CD4-positive lymphocytes, the blood contains an additional subgroup of white blood cells, that are also marked with the CD4 antibody, namely, monocytes. This subgroup interferes with obtaining reliable data on the CD4-positive cells, because these cells are also included in the mass of cells that are attracted by the magnet. The total mass of CD4-positive cells picked up by the magnet thus includes CD4-positive lymphocytes and CD4-positive monocytes. This interfering subgroup can be taken into consideration when determining the count of CD4-positive cells quite simply by taking a second blood sample, marking the blood with a monoclonal antibody that attaches only to monocytes and that carries iron particles, filling a second tube with this sample, and using the magnet to drag the marked monocytes to the narrow end of the tube. All monocytes, exclusively, have now been moved to the end of the tube with the magnet and are clearly visible as a compact mass. Here, too, a fluorescence marker may be added, as well as a lysing agent for destroying the red blood cells.

To obtain the correct value for the diagnostically relevant CD4-positive lymphocytes, the value for these monocytes is simply subtracted from the value for the mass of CD4-positive cells.

It is now possible, knowing the value for the compact mass of CD4-positive cells, to derive the cell count and to determine the concentration of these cells in the blood, because the relationship of compact cell mass to cell count is known.

The method according to the invention also includes steps to determine what percentage of all lymphocytes the CD4-positive T-lymphocytes represent. In this step, a monoclonal antibody that marks all lymphocytes and that also carries iron particles is added to a third blood sample, which is then incubated and then filled into a third test tube. All lymphocytes, both CD negative and CD4-positive, can now be moved to the end of the tube with the magnet and their mass determined. This mass represents the concentration of all lymphocytes in the blood.

### BRIEF DESCRIPTION OF THE DRAWINGS

The present invention is described with reference to the accompanying drawings. In the drawings, like reference numbers indicate identical or functionally similar elements. The drawings are not drawn to scale.

FIG. 1 is a longitudinal cross-section of a tube.

FIG. 2 is a side view of a tube rack/reader, fitted with a test tube.

FIG. 3 is a perspective view of a tube rack/reader, fitted with several test tubes.

FIG. 4 is a perspective view of the rear side of the tube rack/reader.

FIG. 5 is a side view of a second embodiment of a tube rack/reader, fitted with one test tube.

FIG. 6 is a side view of a third embodiment of a tube rack/reader, that holds the test tube in a different orientation than that of the device of FIG. 5.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

The method according to the invention is a way of determining the mass and concentration of particles or cells of interest in a mixed population of cells, preferably the population of cells found in a blood sample. The cells of interest are marked with a monoclonal antibody that is coupled with iron particles and a magnet is used to attract the cells of interest and move them to a location in a test tube, separate from the rest of the blood cells.

The steps of the method according to the invention for preparing the patient's blood for analysis are as follows:

An antibody that is marked with colloidal iron is added to a blood sample and the sample incubated for approximately 10 to 15 minutes. Such antibodies are commercially available in the marketplace. This incubated blood sample is put into a test tube. A magnet is used to attract the marked cells, i.e., the cells of interest to which the CD4 antibodies with the iron particles have attached. By moving the magnet along the test tube, the cells of interest are separated out from the rest of the blood cells, which remain unattracted by the magnet. A line or graduated scale is used to determine the volume of the cells of interest. The scale may be lines drawn or etched directly on the tube or may be a provided on a surface on a rack that holds the tube and that is aligned with the tube. The scale enables one to determine the total volume of the cells of interest, in this case, CD4-positive cells, and also the percentage of the volume of blood that these cells make out.

It is extremely difficult, if not impossible, to recognize the compact mass of CD4 cells without using some type of additional means of making the cells visible. That's because, essentially, blood contains large amounts of other cells, such as, for example, red blood cells, that make it difficult to recognize the marked mass of leucocytes. For this reason, the method according to the invention includes steps to improve the ability to determine the volume of the cells of interest. First step is to mark the blood with two different antibodies, with the antibody that carries the iron particle and with a second antibody that also marks the CD4 antigen, and which, instead of carrying the iron particle, is coupled with a fluorescent dye. In this way, each CD4-positive cell is doubly marked, one part of the epitope binds with the antibody carrying the iron particle and one part of the epitope of the same cell binds with the antibody marked with the fluorescent dye. After incubating the blood, the iron-particle-carrying CD4-positive cells are moved to the end of the tube with the magnet. A light that excites the fluorescence is directed at the mass of cells, which are then made clearly visible.

The ability to recognize the compact mass of CD4 cells at the end of the tube may also be optionally improved by selectively destroying the red blood cells with a lysing reagent. The blood in the clear tube then becomes transparent and the mass of CD4 cells is then easier to recognize. An example of such a lysing agent is “CyLyse”, marketed to the company Partec in Germany.

The inventive method requires the use of test tubes T and a test tube rack/reader 4, which are described below and shown in the accompanying figures. The compact mass of the cells to be counted in the blood is relatively small, and so, tubes T that have an area with a large inner diameter and a measuring area with a small diameter are used. The tube T has a capillary section 2 and a wide section 1. To gather the marked cells, the magnet is moved along the test tube T in the axial direction, from the wide section 1 to narrow section 2 and then left at the top of the tube, thereby holding the cells that contain the iron particle there. The rest of the cells, actually interfering cells, most of all the red blood cells and unmarked white blood cells, sink down to the bottom of the tube of their own accord, because of their higher density than that of the fluid medium. The compact mass of the CD4-positive cells that is to be counted is then separated out and clearly visible. This procedure has the advantage that it is not necessary to use the lysing reagent.

The otherwise interfering cells wander within minutes down the tube, out of the area of the compact mass of marked cells, while, at the same time, the cells of interest are held by the magnet. The mass of cells to be counted is now visible, even without fluorescence excitation. The use of one variation over the other of the method is selectively based on the particular measurement to be done or the type of cells or particles that are to be ascertained.

In addition to CD4-positive lymphocytes, the blood contains an additional subgroup of white blood cells, that are also marked with the CD4 antibody, namely, monocytes. These monocytes interfere with obtaining reliable information on the CD4-positive cells, because they are also attracted by the magnet. The total mass of CD4-positive cells thus includes the CD4-positive lymphocytes and CD4-positive monocytes. Because of that, the method according to the invention includes a correction. The correction is done quite simply by filling a second tube with a sample of the same patient blood. This second blood sample is then, in advance and independently of the CD4 blood sample, marked with a monoclonal antibody that binds only to monocytes. If such an antibody is used, one that carries iron particles, then all monocytes, exclusively, can be moved to the end of the tube with the magnet. Here, too, additional aids, such as adding a fluorescence marker or a lysing reagent to destroy red blood cells, as described above, may also be used.

The value for the mass of monocytes that is then determined is then subtracted from the value of the mass of cells of interest in the first test tube, to obtain a value for the mass of the CD4-positive cells. This gives the correct value for the diagnostically relevant CD4-positive lymphocytes. A calibration is done to determine the relationship of the compact cell mass to cell count and, thus, it is possible to derive the cell count from the value for the compact mass of cells and to determine the concentration of the CD4-positive cells in the blood.

The method may be advantageously expanded to enable a determination of the percentage of all lymphocytes that the CD4-positive T-lymphocytes represent. The percentage of CD4-positive lymphocytes is of particularly great importance for young patients. To determine this percentage, a third blood sample is incubated with a monoclonal antibody, one that marks all lymphocytes and is carries iron particles, and is then filled into a third test tube. All lymphocytes, both CD-negative and CD4-positive, may now be moved to the narrow section of the tube with the magnet and their mass be determined. This mass represents after calibration (which is to be done once by the manufacturer of the test device) the concentration of all lymphocytes in the blood.

The method according to the invention makes it possible to obtain for therapeutic purposes, in a simple manner and with particularly simple measuring devices, measurement values that allow the absolute number of the CD4-positive lymphocytes (concentration in blood) and the percentage of the CD4-positive lymphocytes of the total amount of lymphocytes to be derived.

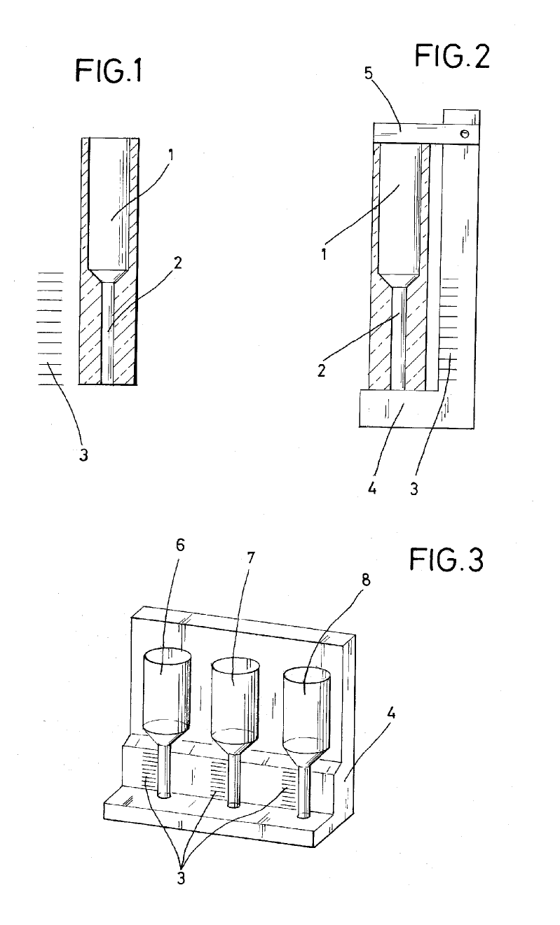

FIG. 1 shows a tube T that includes a first area 1 having a wide diameter that holds a large volume of blood and a second area 2 that is a capillary tube with a much narrower diameter than that of the first area 1. The tube T so constructed has the advantage that it is very compact and has a relatively shorter length, compared to a tube having a narrow inner diameter over the entire length of the tube. The cells of interest in the tube T, i.e., cells that are marked with the iron particles, are attracted by a magnet that is moved along the outside of the tube and guided from the wide section 1 to the narrow section 2. The compact mass of cells fills a greater length of the capillary section 2, which makes it easier to determine a reliable value. The small diameter of the capillary 2 and the corresponding longer stretch of it that is filled with the marked cells result in stretches within the capillary that are filled with relatively different cells, so that is easy to visually read the cells and differentiate them.

The tube T is filled completely with blood and, because the entire inner volume is known, it is possible to determine an absolute cell count by determining the concentration or cell mass. After filling, the tube is placed in a tube holder, which seals the lower end of the tube. Due to capillary action, the narrower capillary portion 2 of the tube fills itself completely. The iron-particle-carrying cells are then guided toward the capillary section 2 with the magnet and collected there as a mass of cells. An example of a suitable magnet is a magnet from the company Dyna that is commercially available. The magnet may be moved manually, or it may be connected to a small electric motor, in which case the magnet moves automatically along the tube from the wide to the narrow section.

Assuming that a test is being done that requires the use of the three tubes, as described above, the procedure can be done for all three tubes in parallel and at the same time.

FIG. 2 illustrates a tube rack/reader 4, referred to hereinafter frequently simply as a tube rack 4. The tube T is clamped into the rack 4, such that the tube is closed at the lower end. A measuring scale 3 is provided on the rack 4, thus enabling one to determine a value for the contents of the tube T. Providing the measuring scale 3 on the tube rack/reader 4, rather than on the tube itself, has the advantage that the tube T may be produced very economically, because it need not be marked with measurement lines.

A cover 5, typically constructed as a snap lid, presses the tube T toward the floor of the rack 4, and prevents blood from seeping out.

FIG. 3 is a schematic illustration of the complete tube rack/reader 4. This rack 4 is designed to accept the three tubes T that are necessary for the complete test. The three tubes T include a first tube 6 for all CD4-positive leucocyte cells, a second tube 7 for the CD4-positive monocyte cells, and a third tube 8 for the overall number of lymphocytes.

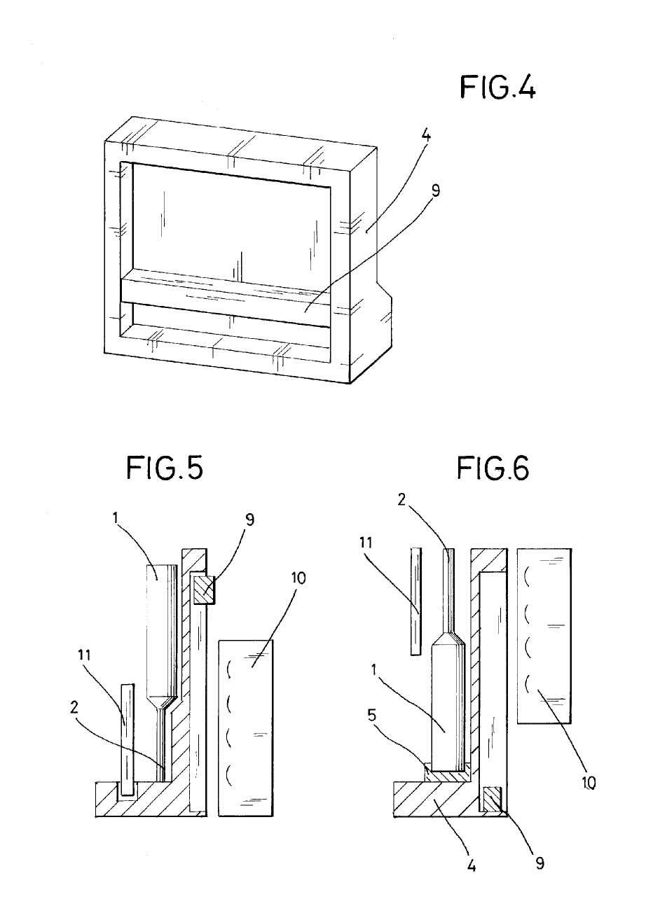

FIG. 4 shows the rack 4 from the rear and illustrates the placement of a magnet 9. This magnet 9 is moved from the top of the rack 4 down or, alternatively, from the bottom up, depending on the orientation of the tubes 6, 7, 8, as closely as possible to the tubes, in order to guide the marked cells and to fix them as a compact mass. The tube rack/reader 4 may be constructed as, viewed from the front and read, an open, generally rectangular frame, and with an L-shape viewed from the side, as shown in the figures. The magnet 9 may be provided as a loose, freely movable element, so that the magnet 9 may be placed directly up against the tubes 6, 7, 8. It is foreseeably an advantage, though, to provide the magnet 9 in the rack 4, so that it cannot be lost, i.e., is always in position, ready to be used.

The tube rack/reader 4 may be embodied as an open frame, with measurement scales 3 placed advantageously in the rack, between the narrower portions 2 of the tubes. The scales 3 may be placed on a transparent or opaque surface. If the tube rack/reader 4 is constructed to have a rear wall, then it may be advantageous to have the wall be transparent, i.e., see-though, or at least translucent, i.e., with illumination shining through, in order to simplify reading or recording the mass of cells. In this case, the measurement scales 3 are preferably mounted on the rear side of this wall.

If only the concentration of the CD4-positive cells is to be determined, then the tube rack/reader 4 is fitted with only two tubes T.

The tubes 6, 7, 8 may have different shapes, as a way of avoiding a mix-up. For example, they may have different lengths or diameters, with the particular shapes keyed to particular retainer recesses or spaces on the rack 4, so that each tube 6, 7, 8 is able to be fitted only in its particular space. The magnet 9 is mounted in the tube rack/reader 4, such that it may be moved manually or by an electric motor.

The movement of the magnet 9, when moved manually, is controlled by the person moving the magnet. Alternatively, the magnet 9 may also be operated by means of a turn crank with a limiting gear, so as to limit the speed of the magnet 9 as it is guided along the tubes 6, 7, 8. In this way, as also when an electric motor is used, it is possible to be sure that the speed of the magnet 9 is such, that all correspondingly marked cells are reliably attracted by the magnetic force and transported into the capillary portion 2 of the respective tube T. The turn crank may serve to mechanically drive the magnet 9 or to operate a dynamo, so that, even without an external power source, it is possible to use an electro-motor to move the magnet 9 at a pre-determined speed.

The measurement scales 3 for the three different tubes 6, 7, 8 are preferably mounted on the tube rack/reader 4. Instead of using individual tubes, an injection molded component having three hollow chambers that correspond to the chambers in the tube T described above may be used for holding the blood samples. In this case, each of the chambers has a large-volume chamber with a connecting capillary tube.

FIG. 5 illustrates a set up to observe cells marked with fluorescence. An LED device 10 is placed so as to excite the fluorescence markers in the cells that are gathered in the capillary portion 2 of the tubes T. A blocking filter 11 allows only the fluorescence light, which has a longer wave length, to pass through it, so that the cells with fluorescence are easily seen and distinguishable from other cells.

The calibration carried out by the manufacturer allows one to directly determine the respective cell concentrations by means of the respective measurement scales 3. This is possible, because there is a direct relationship between the volume of densely packed cells of a known volume of blood and the cell count.

FIG. 6 illustrates a second embodiment of the tube rack/reader 4, in which the tubes T are held in a different orientation than that shown in the preceding figures. In this embodiment, the tube T is placed in the rack 4 so that the first area 1 with the wide diameter is at the lower end and the capillary section 2 with the narrow diameter at the upper end. The lower end of the tube T is covered with the cap or lid 5, to prevent blood from seeping out. In this embodiment, the magnet 9 is moved from the bottom toward the top. Within minutes, the uninteresting, non-marked cells sink toward the bottom and the interesting cells are moved toward the top into the capillary section 2 and fixed there by means of the magnet 9. There, the entire volume can be read, with or without fluorescence. The LED device 10 and the blocking filter 11 are shown here, for easiest possible detection of the fluorescence light, although it is understood that these elements are not absolutely necessary to carry out the method according to the invention.

Deviating from the embodiments shown, the tubes T may be constructed such, that the capillary portion 2 is not located along the central axis of the tube, but is laterally offset. Thus, the tube may have, for example, a rear inner tube wall that extends in a straight line along the entire height of the tube. In other words, the inner surfaces of the rear walls of the first area 1 and the second area 2 are aligned in a straight line. This ensures that the entire length of the filled tube lies close to the place along which the magnet 9 is moved up and down. As a result, the strongest possible magnetic field is able to act on the cells to be counted all along the length of the tube.

As an alternative, the tubes T may have the shape shown in the figures, but instead of guiding the magnet 9 along a linear path up and down, the magnet 9 may be guided along a path that corresponds to the profile of the tube, so as to hold the magnet 9 in a path that is optimally close to each section 1, 2 of the tube T.

An embodiment that is only slightly more complex technically includes an optical detection device for automatic detection of the marked cells. The advantage of this embodiment is that it precludes human error. For example, automatic detection may be accomplished by means of electronic image detection or image analysis. Suitable procedures for this are relatively simply to program and to implement technically. Such programs are widely used or known, for example, as applications in a mobile phone that has a camera, and also as bar code scanners.

The optical detection device has a camera, for example, one such as is used in mobile phones. The camera is directed to the area of at least one tube T that contains the cells of interest, and preferably at the corresponding areas of all tubes T held in the tube rack/reader 4. Preferably, a defined space or retainer for positioning the camera is provided on the tube rack/reader 4, so that the distance, the detection area, and the observation angle of the camera relative to the respective tubes T is always the same.

The optical detection device has electronic circuitry, for example, the electronics of the mentioned mobile phone, which is used to run the program application mentioned above. Automatic image detection or image analysis of the images recorded by the camera is done with this electronic circuitry. In this way, the volume of marked cells is detected. Also, when the optical detection device is appropriately calibrated, it is possible, based on the known distance between the camera and the tube T, to gain information about the volume of the marked cells, even without the use of a measurement scale. Even without such a calibration, it is possible to obtain reliable information on the volume of the marked cells, if, in the image analysis, not only the marked cells are taken into consideration, but also the mentioned measurement scale, which can be provided next to or on the tube, so that the fill level of the marked cells within the tube may be automatically assigned a value on the scale.

The optical detection device has a display, here again, think of the display on the mobile phone, by means of which the volume of detected cells or the detected corresponding scale value may be displayed to the operator of the device, so that the operator then may enter the value in a chart, a patient's file, or similar. The representation of the automatically detected scale value is preferably done in numeric form.

### ADVANTAGES OF THE INVENTION

The method according to the invention for determining the concentration of a known sample volume requires very little lab resources to mark the cells. To further simplify the method, the antibodies that are intended to be used in the test tubes may be stored as freeze-dried or lyophilized antibodies. The antibodies, which normally require scientific storage in a wet state at 4 to 6 degrees C., may now be kept for any length of time, even at room temperature.

With this method, lab work is reduced to filling the tubes with blood, incubating them for approximately 10 to 15 minutes in the dark, and then placing the tubes in the tube rack/reader. This simple procedure does not require a well-equipped laboratory, nor does it require particularly extensive training of the lab personnel. No complex electronic equipment is needed, as is the case with the method used to date to determine the CD4 concentration. Also, the method does not depend on electric power.

The method may be carried out as a “point of care” method anywhere, where the affected persons live. Patients no longer need to travel to centralized laboratories. Further advantages of the method include the relatively low cost per test. The extremely high costs for the acquisition of a flow-through cytometer drop away, as do the high operating costs for these technologically sophisticated devices. The follow-up costs, particularly, for the service, maintenance, and repair are eliminated. The cost for producing the test tubes T according to the invention is in the range of Euro cents. The test tubes T are single-use articles made of glass or plastic. Extensive training of lab personnel is not necessary.

The tube rack/reader 4 according to the invention does not contain any sensitive electronic and/or optical components, such as are needed in the flow-through cytophotometer. The tube rack/reader with its movable magnet, possibly with a battery-powered LED light source and the light filter for observing fluorescence, costs only a few Euros; it has an unlimited service life and requires absolutely no maintenance or repair resources.

A further advantage of the method of the very small device, which can be called a mini-apparatus, is that it requires no complex logistics to bring the test to the patient.

It is understood that the embodiments described herein are merely illustrative of the present invention. Variations in the method and the construction of the test tubes and tube rack/reader according to the invention may be contemplated by one skilled in the art without limiting the intended scope of the invention herein disclosed and as defined by the following claims.

What is claimed is:

1. A method of determining the mass and concentration of particles or cells of interest from a mixed population of cells, preferably from a patient blood sample, the method comprising the step of:
a) marking the cells of interest in the blood sample with a monoclonal antibody that carries colloidal iron.

2. The method of claim 1 further comprising the steps of:
b) placing the blood sample in a test tube that has at least a capillary section; and
c) moving a magnet along the test tube, so as to attract the cells of interest to a compact mass in the capillary section.

3. The method of claim 1, further comprising the step of:
d) marking the cells of interest additionally with a fluorescing antibody.

4. The method of claim 1, further comprising the step of:
e) lysing red blood cells in the blood sample.

5. The method of claim 1, further comprising the step of:
f) treating the blood sample with CD4-specific antibodies.

6. The method of claim 1, further comprising the step of:
g) treating the blood sample with monocyte-specific antibodies.

7. The method of claim 1, further comprising the step of:
h) treating the blood sample with lymphocyte-specific antibodies.

8. The method of claim 2, wherein the blood sample includes a first blood sample treated with CD4-specific antibody, a second blood sample treated with monocyte-specific antibodies, and a third blood sample treated with lymphocyte-specific antibodies, the method further comprising the steps of:
i) providing three test tubes; and
j) filling a first test tube with the first blood sample, a second test tube with the second blood sample, and a third test tube with the third blood sample.

9. The method of claim 1 further comprising the step of:
k) providing the monoclonal antibody in lyophilized form; and
I) placing the monoclonal antibody in the test tube, before the blood sample is filled into the test tube.

10. The method of claim 1 further comprising the step of:
m) placing the test tube into a tube rack/reader.

11. The method of claim 10 further comprising the steps of:
n) placing the test tube into the tube rack/reader with the capillary section at an upper end of the test tube;
o) gather the cells of interest with the magnet and moving them up to the capillary section, wherein interfering cells or particles sink to a lower end of the test tube; and
p) beginning an optical image detection of the gathered cells of interest after the interfering cells or particles have sunk to the lower end.

12. A tube rack/reader for use in a method of determining a mass and a concentration of particles or cells of interest in a mixed population of cells, preferably from a patient blood sample, the tube rack/reader comprising:
a test tube having a capillary section; and
a rack for holding at least one of the test tube with the capillary section.

13. The tube rack/reader of claim 12 further comprising:
a light source suitable for fluorescence excitation;
wherein the rack includes a frame that has a rear wall in front of which the test tube is held, the rear wall allowing light to pass through; and
wherein the light source is placed behind the rear wall.

14. The tube rack/reader of claim 13 further comprising:
a blocking filter that only allows passage of light having a greater wavelength than light from the light source;
wherein the blocking filter is placed in front of the test tube.

15. The tube rack/reader of claim 12 further comprising:
a measuring scale;
wherein the capillary section of the test tube has a capillary length and wherein the measuring scale is provided at a height that corresponds to the capillary length.

16. The tube rack/reader of claim 12 further comprising:
a cover that closes an open end of the test tube.

17. The tube rack/reader of claim 12 wherein the rack holds the test tube in an orientation in which the capillary section is at the upper end of the test tube.

18. The tube rack/reader of claim 12, wherein the test tube includes three test tubes and the rack is constructed to hold the three test tubes at the same time.

19. The tube rack/reader of claim 12 further comprising:
an optical image detector that includes a camera, electronic circuitry, and a display;
wherein the camera is directed toward the area of the test tube that holds the cells of interest;
wherein the electronic circuitry is used to automatically record and evaluate an image that encompasses the volume of the cell of interest; and
wherein the display displays the volume calculated by automatically by the image detection of the cells of interest and/or displays an automatically recorded measurement scale value that corresponds to the volume of the cells of interest.
